(19) United States
(12) Patent Application Publication
(10) Pub. No.: US 2008/0201641 A1
(43) Pub. Date: Aug. 21, 2008

(54) Method And The Associated Mechanism For 3-D Simulation Stored-Image Database-Driven Spectacle Frame Fitting Services Over Public Network

(76) Inventor: Yiling Xie, Arcadia, CA (US)

Correspondence Address: LAW OFFICES OF J.F. LEE, 17800 CASTLETON STREET, SUITE 383, CITY OF INDUSTRY, CA, 91748, US

(21) Appl. No.: 11/677,141

(22) Filed: Feb. 21, 2007

(51) Int. Cl. G06F 3/00; A61B 3/00

(52) U.S. Cl. 715/733; 351/200

(57) ABSTRACT

A method of spectacle frame 3-D simulation fitting over public network, such as Internet, based upon database of product information and digitized user images as acquired via devices connected to computer. Particularly, consumers can take advantage of present method to choose spectacle frames from wide variety of selections, expeditiously by the use of public computer network (Internet). Consumers may use digital cameras, network cameras or scanned photos to submit facial image, and by way of calibration steps and additional side-view images of the consumers when test-fitting spectacle frames, simulated 3-D presentation of a consumer wearing the intended spectacle frame can be viewed for purpose of fitting and purchasing over the network. With the accessibility and availability of the Internet, just a few clicks on the mouse enable the consumers to choose spectacle frames of their preference and in a way that affords wide selections at low costs and easy access.

### FIELD OF THE INVENTION

The present invention relates generally to a method of spectacle frame test-fitting over public network such as the Internet based upon database of product information and digitized user images as acquired via devices connected to computers. Particularly, consumers can take advantage of present method to choose spectacle frames from wide variety of selections, expeditiously by the use of public computer network (Internet). With the accessibility and near universal availability of the Internet, just a few clicks on the mouse enable the consumers to choose spectacle frames of their preference and in a way that affords wide selections at low costs and easy access and promote the efficiency in the spectacle fitting process.

### BACKGROUND AND PRIOR ART

People wearing glasses or spectacle frames prefer to try on different styles of frames. Before making purchases, test-fitting spectacle frames include checking out the availability and styles of frames on the market, to see what would fit nicely with the facial features of a person wearing glasses. In any given store where a consumer visits, only limited amount of frames can be made available to people who come in to test fit. As a result, a consumer will not have the chance to test fit some styles that are not in the stores visited.

A store, on the other hand, may lose a sale simply because it is not able to provide the opportunity to the consumer to test fit certain style or model of frames that are best suited for a consumer.

The recent development of Internet and its universal accessibility due to the popularity of personal computers in everyday households, and other sites of connectivity such as Internet Café, enable the huge growth in Internet commerce, so-called e-commerce. A substantial portion of the e-commerce today actually involves the selling of lens and spectacle frames for lenses of different purpose. However, despite the accessibility of such spectacle frames over the Internet, there has not been a viable Internet test-fitting system and method for consumers to try on the wide variety of different styles to fit facial features and other individual preference before making the purchase.

Although programmers can put together software programs that can fit a facial image to a spectacle frame image, there is no functional feature to change or scale the size of the spectacle images, relative to that of a face.

Present invention differs and improves over prior art patent Fay U.S. Pat. No. 5,983,201 (Fay 201) in that consumers, according to Fay 201, need to have their frames fitted in a remote electronic store (RES). All the limitations associated with RES are not present in present invention, including the travel to and from consumers' place to RES and the diagnostic procedures and restrictions employed by RES.

Additionally, opticians regularly suggest the check for eyesight, and the consequential fitting of updated prescription and frames also entails more inconvenience associated with RES as stated in Fay 201.

U.S. Pat. No. 6,791,584,(“584 Patent”) issued to the same inventor of present application, is disclosed to resolve the problem associated with fitting computer stored images of spectacle frames to scalable human faces, making it easier to fit frames to faces via the use of stored images in computers. However, 584 Patent provides the virtual effect of test-fitting only to the user's front facial image. How the legs of spectacle frame will be seen by people, in a 3-D environment, is an important consideration to potential, especially in the case of purchasing a pair of sunglasses and where sunglasses are worn over optical lenses as shelter frame glasses.

### SUMMARY AND OBJECT OF THE INVENTION

Present invention builds upon applicant's prior technological disclosure, to implement a 3-D simulation, database-driven stored-image spectacle frame fitting method and its associated mechanism, enabling consumers to make purchase decisions of spectacle frames and products over the public Internet by selectively “trying on” different spectacle frames on the facial image, saving the trouble of actually fitting on real spectacle frames by visiting multiple stores.

To achieve the goal stated above, the database-driven stored-image spectacle frame fitting method involves the following steps:

(a) Produce the frontal view and side view s of spectacle frames having open frame legs and derive the digital image of said frontal/side views after the background and the optical lenses are made transparent and said side view images are based upon preset angles of said frame when rotated about the center of said frame, with each of said side view frame images further divided into images showing the legs being obscured as perceived due to the angle of rotation; and,

(b) Enter the size parameters and other relevant attributes of said digital images when they are stored into computer databases.

(c) Set up a calibration paste-over device, so that said paste-over device produce a benchmark ruler on the display device and that said benchmark ruler contains two marking points that can be used as the alignment/matching reference points relative to facial images when displayed.

The benchmark ruler can be moved, dragged, expanded and contracted, relative to the facial image, so that the marking points can be set to superimposed upon selected marking points (such as centers of pupils) on facial images, resulting in the correct size calculation of face relative to the stored images of the spectacle frames, based upon the attributes of height and width of spectacle frames that have been pre-loaded into database.

The ratio of frame size to facial image size is then used by other paste-over device to put all other spectacle frames as chosen by consumers to “try on” to the facial images, allowing consumers to see if he/she likes the simulated “looks” of “wearing” the selected spectacle frames, after the computer has correctly set the size ratio of all other spectacle frame images, due to the benchmark superimposition process described hereinabove. The marking points on the facial image, when side view images are presented, can be used to measure the rotation angle of the image, so that computer can pick one of the side images of the stored spectacle frames that would produce the best fit for the angel of the side facial image, resulting in a 3-D simulation of a person “wearing” a spectacle frame as viewed from multiple angles.

Said calibration paste-over device further includes a crop-block used to the ear position on a facial image, where the crop-block, as a first step in the crop-and-paste-over, is to crop out the ear of a facial image. As a second step, the selected spectacle frame image is then pasted to the facial image where the ear has been cropped out. As a third step, the cropped out ear portion is then pasted back to its original place.

The attributes being stored into computer database, as stated herein, include the width and height of the spectacle frame and the mid points of frame as measured on the frame either vertically and horizontally.

A further treatment, as disclosed and claimed in present invention, is to add crescent-shaped reflective color effect (available in popular photo/graphics software applications and programs) to the opposite and corresponding spots on the two lenses, or the area on the spectacle frame defining two lenses. Coupled with the treatment of fading (color or gray level) processing, the reflective color effect is made more life-like. The lower portion or the underside of the spectacle frame image can also be superimposed with shade-lines and shades of gray, to enhance the effect of color fading. Additionally, the image of the spectacle frame and the area containing lenses on the spectacle frame and the background, after having been made transparent, can be made to have 3-D features to give better contextual view for consumers.

To achieve the goal stated above, present invention further disclosed a spectacle frame 3-D simulation fitting mechanism for choosing optical products over the public network. Such mechanism is made up with:

A choice center containing a computer database stored with images of spectacle frames, so that the display outlets of said computer database can be accessed via the use of public network, such as Internet, wherein the images of spectacle frame may be viewed and selected for manipulation by users for spectacle frame fitting purpose. Each of the digital image of spectacle frame contains frontal and side images of said frame and will be obtained by the process described below:

Produce the frontal view and side views of spectacle frames having open frame legs and derive the digital image of said frontal/side views after the background and the optical lenses are made transparent and said side view images are based upon preset angles of said frame when rotated about the center of said frame, with each of said side view frame images further divided into two images showing the legs being obscured as perceived due to the angle of rotation; Enter the size parameters and other relevant attributes of said digital images when they are stored into computer databases;

Set up a transmitting/operating platform containing display device that is connected to above-said computer database via communication protocol, wherein users may transmit front and side facial images from said platform and have the images shown on the display device;

Set up a calibration paste-over device containing a benchmark ruler on the display with two marking points that can be used as the alignment/matching reference points relative to facial images when displayed. The benchmark ruler can be moved, dragged, expanded and contracted, relative to the facial image, so that the marking points can be set to superimposed upon selected marking points (such as centers of pupils) on facial images, resulting in the correct size calculation of face relative to the stored images of the spectacle frames, based upon the attributes of height and width of spectacle frames that have been pre-loaded into database.

When the cropping and pasting back is applied to the side view facial image of a consumer, the ear portion of the facial image will be subdivided into three smaller images in the first crop-out step. As a second step, and ear image of a consumer being fitted with a spectacle frame would remain on the display device. As a third step, the spectacle frame side view where part of a leg is covered will be pasted over the position relative to the ear on the facial image of a consumer. As a fourth step, the user image containing no ear or ear shape will be pasted back to the original place. As a fifth step, the spectacle frame side view containing no covered leg will be pasted over the image containing no ear or ear shape on the user's facial image. As a sixth step, the cropped out ear image will be pasted back to its original place.

Preferably, the marking points on the facial images are the centers of pupils. The distance of the two pupil marking points may be obtained at time of optician measurement; users may also obtain the distance figure by self-measuring. Alternatively, a ruler may be used in a facial image acquisition process, by placing a vertical/horizontal ruler alongside a user's face. Marking points can also be the pair of points defining the two sides of a person's mouth; they can also be the points defining the vertical distance between eyes and nose.

Similarly, a vertical/horizontal ruler may be placed alongside a user's face when obtaining the distance of marking points other than the two pupils.

Marking points can also be the spectacle frame already worn on a user's face, especially in the case where the user is choosing to purchase a shelter frame to wear over the optical spectacle frames (near-sightedness or far-sightedness). The measurement of marking points based upon a person already wearing optical spectacle lenses is straightforward. The center points in the pupils and other points such as the vertical central line drawn along the nose section can serve as the measurement baseline, for moving and dragging rulers for aligning spectacle images and calculating size ratio.

The database-driven stored-image spectacle frame fitting method and mechanism as described herein allows users to take advantage of digital cameras, web cam and/or scanners, via the easiness of public network such as Internet, and to have the opportunity of seeing own looks of how well some chosen spectacle frames of different styles/colors would fit on one's own face.

Following the calibration steps described herein, the ratio of spectacle frame size to the facial image size can be determined by the alignment/superimposition of marking points on the benchmark ruler and those of the facial image. The automatic process of applying the same ratio to all other chosen spectacle frame images, by the simple operation of moving and dragging computer mouse and cursor on display device as stated herein, reduces the user trouble of physical travel to optician's office, traditional retail store, or the RES in Fay 201, and the fitting process is made more painless and efficient.

Present invention provides a cost-effective solution to the spectacle frame try-on and purchase process, by the use of computer database and the publicly available Internet and also may be used by optical industry as auxiliary infrastructure for research and promoting better consumer optical products.

### BRIEF DESCRIPTION OF THE DRAWINGS

The accompanying drawings, which are incorporated in and constitute a part of this specification, illustrate the preferred embodiment of the invention and together with the description, serve to explain the principles of the invention. A brief description of the drawings is as follows:

FIG. 1 shows a first block diagram for major system structure of a spectacle frame fitting method in accordance with the disclosure of present invention.

FIG. 2 shows the flow chart of the optical product buying process over the public network in accordance with the disclosure of present invention.

FIG. 3 shows the first step of producing the side digital image of a spectacle frame for storing into computer database in accordance with the disclosure of present invention.

FIG. 4 shows the second step of producing the side digital image of a spectacle frame for storing into computer database in accordance with the disclosure of present invention.

FIG. 5 shows the third step of producing the side digital image of a spectacle frame for storing into computer database in accordance with the disclosure of present invention.

FIG. 6 shows the fourth step of producing the side digital image of a spectacle frame for storing into computer database in accordance with the disclosure of present invention.

FIG. 7 shows only the user facial image when the spectacle frame fitting mechanism is used in accordance with the disclosure of present invention.

FIG. 8 shows the frontal image of a user along with the bench mark ruler in accordance with the disclosure of present invention.

FIG. 9 shows the side image of a user along with the benchmark ruler in accordance with the disclosure of present invention.

FIG. 10 shows the crop-block portion of a paste-over device, used in the second step of pasting-over by superimposition of digital frame image over the facial image, in accordance with the disclosure of present invention.

FIG. 11 shows the third step of pasting-over by superimposition of digital frame image over the facial image, in accordance with the disclosure of present invention.

FIG. 12 shows the fourth step of pasting-over by superimposition of digital frame image over the facial image, in accordance with the disclosure of present invention.

FIG. 13 shows the fifth step of pasting-over by superimposition of digital frame image over the facial image, in accordance with the disclosure of present invention.

FIG. 14 shows the sixth step of pasting-over by superimposition of digital frame image over the facial image, in accordance with the disclosure of present invention.

FIG. 15 shows the diagram for the spectacle frame fitting mechanism wherein the image is angled in a way to mirror the image in FIG. 9 containing only user image.

FIG. 16 shows the superimposition of digital frame image over the facial image as shown in FIG. 15.

### DETAILED DESCRIPTION OF THE PREFERRED EMBODIMENT

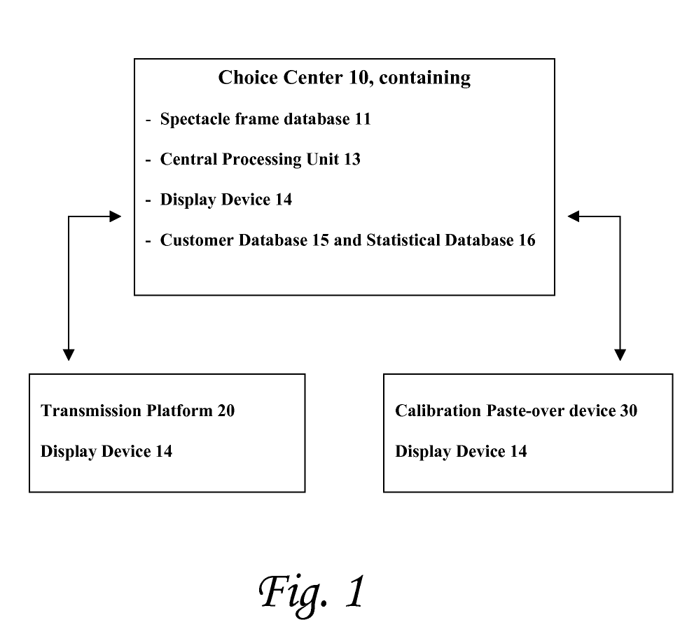

As shown by all the figures, and specifically in FIG. 1, the ideal implementation spectacle frame fitting method and mechanism over the public network, Internet, is composed of a Choice Center 10, a transmission platform 20 and a calibration paste-over device 30. Choice Center 10 includes a spectacle frame database 11, central processing unit (CPU) 13 and a display device 14.

In spectacle frame database 11, different attributes can be pre-set by classifications of manufacturers, brands, genders, materials, shape of frame, colors and varieties of functions. Said database 11 is configured as suitable for access via public network, such as Internet, so that a huge selection of digital frame images 12 are available for consumer to choose for fitting. Said selection of digital frame images 12 contains the real size ratio figures of all the stored spectacle frames.

The image for each and every digital image of the spectacle frame is produced in the following manner:

Produce the frontal view and side view images of spectacle frames having fully open frame legs, as present invention prefers the fully open frame legs that would show the curved portion of the legs. This can be done by a digital camera, a scanner serving as the image in-take device, or any other image acquiring hardware, such as a normal optical camera, which would entail further scanning and digitization.

The background and the area on the spectacle frame containing optical lenses are then made transparent, by the use of commercially available photo or graphics software such as PhotoShop. The transparentization treatment includes making the image of the spectacle frame to appear more like 3-D image in simulation. This can be readily done by many photo-touch-up or graphics software, that normally perform shifting or position translation on the edge or border, and then perform gray lever, brightness and color adjustment. Additionally, the crescent-line-shaped reflective color effect to the opposite and corresponding spots on the two lenses, or the area on the spectacle frame defining two lenses, is used along with the color-fading treatment, to simulate the light-reflection as experienced around optical objection in real life. The color-fading treatment, also called blurry effect treatment, is a form of graphic processing technique available in PhotoShop, and is implemented by a mean-value algorithm. The lower portion or the under surface of a spectacle frame can have shaded lines pasted on, also to achieve the color-fading effect and to make the shadedness look more lifelike.

The side view digital image, as taken by digital camera or other medium, of spectacle frame is obtained when the spectacle frame is placed at some pre-set angles, rotating about and pivoting point selected from the center of the frame. These angles can be 5 or 10 degrees increment within a 90 degree range, to show several side views; each of the angled view spectacle frame image, containing some covered leg portion, will be further divided into two side view digital images.

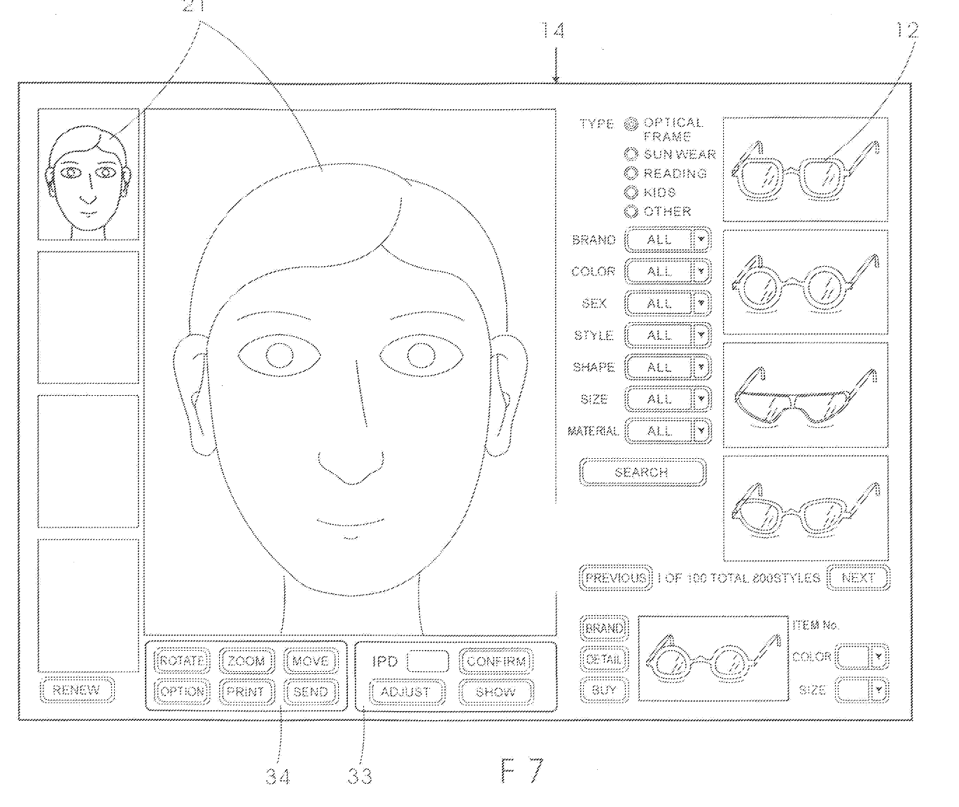

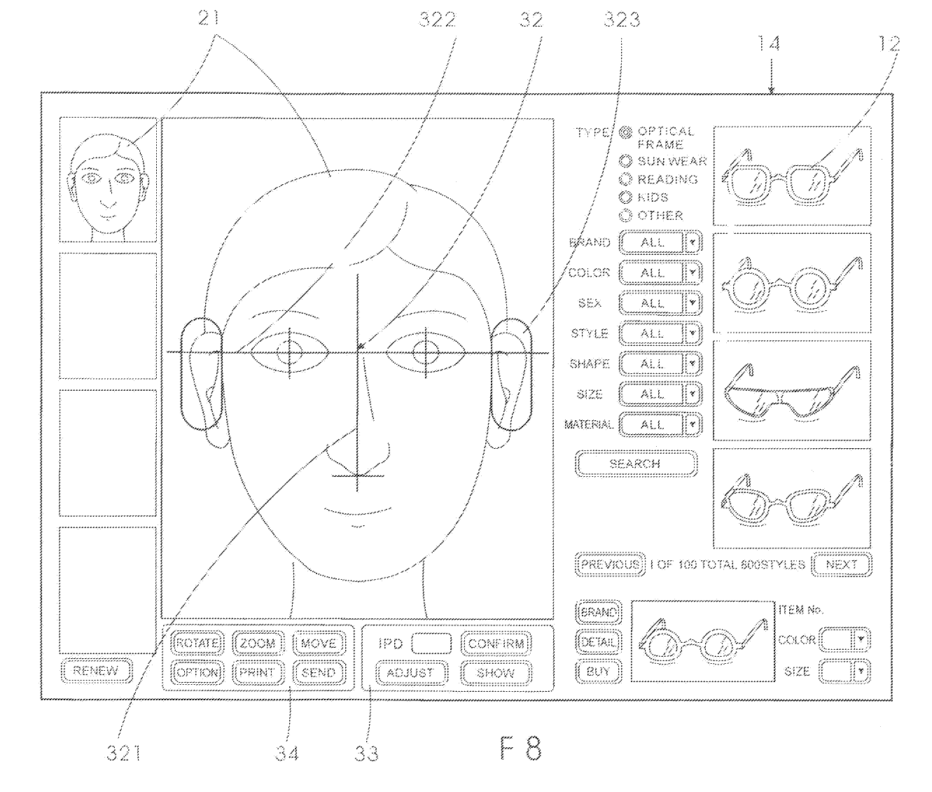

When the digital images of the spectacle frames, as treated by the steps stated above, are stored into the computer database 12, various attributes are also stored, including the horizontal mid point between the spectacle frames, the real size, and the angels of each of the side view images relative to the frontal view, enabling the stored images to have the proper ratio relative to the real spectacle frame. The horizontal and vertical mid points of the spectacle frame, expressed in its real world size measurement, are also stored into the database as the attributes pertaining to the image; as is depicted in FIG. 4 where the measurements of horizontal and vertical lines and mid points are indicated.

Although the depictions contained in present application show the traditional spectacle frames wherein optical glass pieces are fully surrounded by the frame, the disclosure herein, related to scanning and measurement taking also apply to the newer type of spectacle frames where the glass pieces are only half-surrounded or secured to the frame by a few screws or other means without the surrounding frame materials.

The frontal images of spectacle frames to be fitted on to a consumer's face, after the treatment stated above, will appear to have more real-life effect, greatly enhancing the buying experience as consumers look at how the spectacle frames are fitted to their faces.

When spectacle frame digital images is superimposed onto image 21, the reflection effect will be presented on the lenses, and the shades outside the frame on image 12 as appeared on image 21 will give the sense of 3-D virtual reality, close to the experience when people really trying on spectacle frames.

As a result of computer's calculation, the number of stored images in the database can be substantially reduced, such that a 120-degree angle side view image can have its image made from a 30-degree angle image, deriving yet another spectacle frame digital image without the process of image intake and storage. The process can be done during the paste-over operation, and controlled by user via a sub-window on a display device 14.

Transmission platform 20 is suitable for use when users transmit their image 21 via the display device 14 on said platform 20 over the public communication network. Additional side view images o users are also transmitted.

Alternatively, users can transmit their image 21 directly to the display device 14. Image 21 contains two pupils serving as basic benchmark points; calibration paste-over device 30 then aligns the two marking points on benchmark ruler 32 to the two pupils on image 21, and superimpose the image of the spectacle frame as selected by users on image 21, simulating the virtual look of a person wearing a spectacle frame on the face.

The two marking points are based on the same size ratio as users' marking points (two pupils) and the real distance between the two pupils 31. The distance of the two pupils 31 is used to calibrate the ratio for the stored image of spectacle frames to be put on user's face 21 and to insure that the image of the spectacle frame is properly sized to fit the size of a user's face.

When side view images are used for calibration paste-over, the center point of two pupils and the nose tip point will form a vertical line, serving as the basis for calculating real distance when measured using the same ratio previously obtained as stated above.

In accordance with the disclosure of present invention, calibration paste-over device 30 preferably has a moveable vertical plumb line 321, serving as the vertical baseline on display device 14 separating the two pupils 31 on the image of a user. Calibration paste-over device 30 further has a moveable horizontal measurement line 322, serving as the measurable ruler on the face of a user's image and also forming a cross-point with the vertical plumb line 321.

The benchmark ruler 32 in the calibration paste-over device 32 is made up of vertical plumb line 321 and horizontal measurement line 322, where the vertical plumb line 321 is aligned with the vertical marking points on user image 21 and the horizontal measurement line 322 is aligned with the horizontal marking points on user image 21.

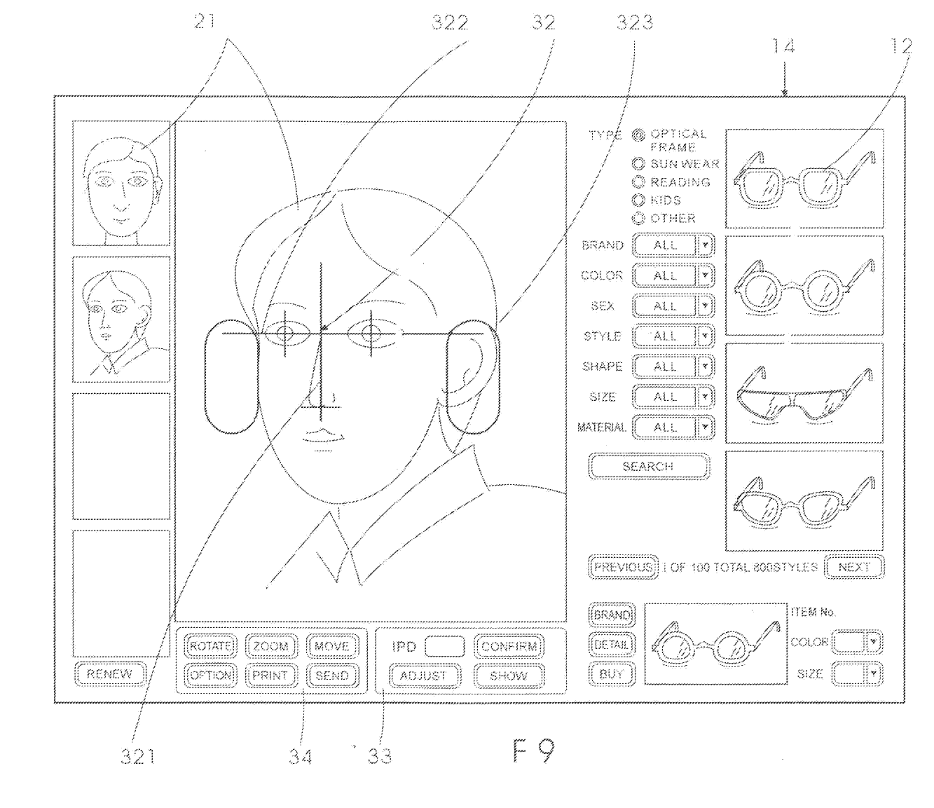

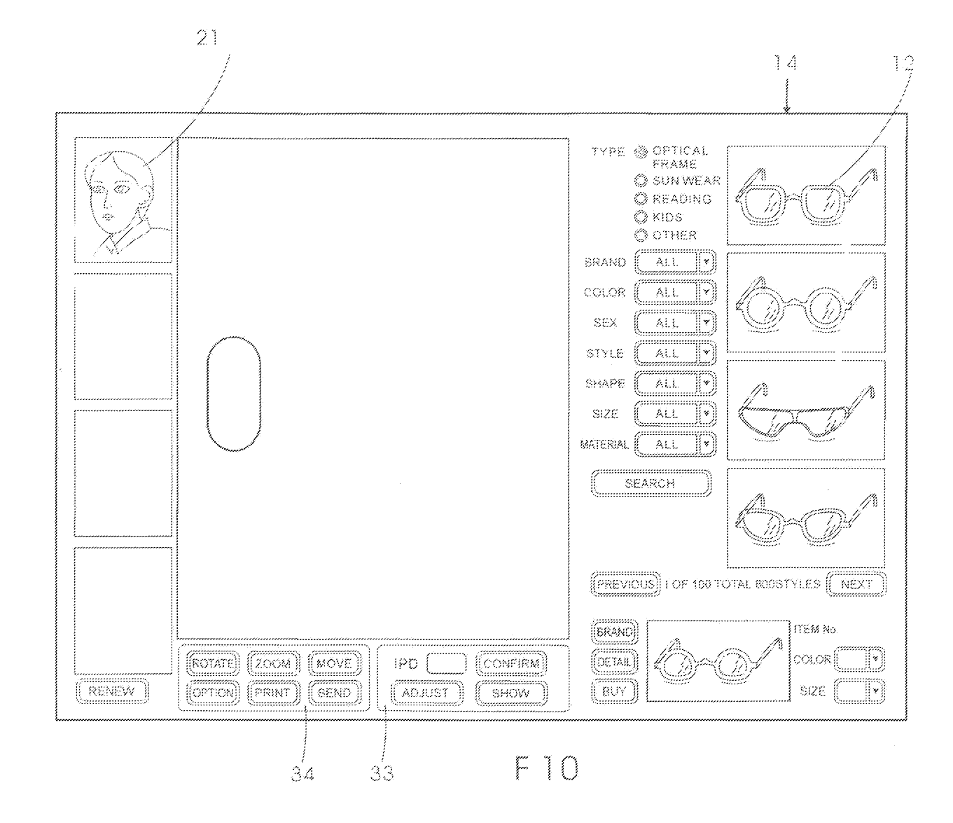

As shown in FIG. 7, the benchmark ruler 32 contains two cross-points made up by the vertical line 321 and horizontal line 322 when the cross-points are dragged to align with the two pupils 31 on the image 21. The distance between the two cross-points, as used by the treatment stated here, can be calculated, even if the figure is not pre-measured.

Benchmark ruler 32 serves primarily as a marker for vertical/horizontal coordinates. The distance between two pupil 31 can be obtained by the calculation of two sets of coordinates measured using the dragging/moving/marking of the vertical line 321 and horizontal line 322 and is further used to ascertain the pupil distance in the situation where side view image of a user's face is presented for spectacle frame fitting. Benchmark ruler 32, along with the horizontal line 322 work together to calculate the pre-set angle of the user image, ensuring the fitting is done properly.

Calibration paste-over device 30 contains an operation window 33 for user to input the real distance between two pupils 31, to work with the benchmark ruler 32. Once the real distance between two pupils is entered, vertical and horizontal lines 322 and 321 can be used to align the cross-points to the two pupils and thus obtaining the size ratio of the face, which is then used by database 11 adjust the displayed ratio of the stored images 12 for the spectacle frames, before the computer performs the superimposition of spectacle frames to the facial image of a user.

In a preferred embodiment of present invention, digital image 12 of spectacle frame remains fixed, whereas the facial image 21 on the transmission platform 20 can be enlarged, contracted, rotated or dragged about for alignment and superimposition of the pupil points, or other marking points. As such, the benchmark ruler 32 also contains fixed value, until the facial image 21 is manipulated (enlarged/contracted/dragged/rotated) to align with selected marking points.

Therefore, the advantage of present invention allows the image of stored spectacle frames to remain fixed during the fitting process, whereas the user facial image 21, as shown on the display device 14 of the transmission platform 21, can be made larger or smaller, or rotation about certain axle, until the two marking points are aligned to the two pupils 31 of the user image.

Alternatively, the image 21 on the transmission platform 21 can be made to remain fixed during the fitting process, whereas the image 12 of the spectacle frame can be made larger or smaller, or rotation about certain axle, until the two marking points are aligned to the two pupils 31 of the user image; or until other selected marking points are aligned to the corresponding marking points on the user image.

As a preferred embodiment of present invention, display device 14 contains a benchmark ruler operation window 33 for setting the distance between two marking points. Other sub-windows that would pop up to ask for display and adjustment of benchmark ruler are within the knowledge of people reasonably skilled in the computer programming field and is not stated here for reason of brevity.

Benchmark ruler 32 can have zoom in/out, shifting movement and rotational movement, to set and calculate distance of any select marked points based upon known points with know distance.

A separate zoom and control window 34 can be employed in display device 21, to allow for the enlargement or reduction of image. Alternatively, other sub-windows can be built to achieve the same purpose and is not stated herein for reason of brevity.

The calibration paste-over device 30 needs to have the real distance between pupils of a user; however, most people do not know this distance. To solve this minor inconvenience, a ruler can be used to be placed alongside the facial image of a user when acquiring the image of a user, for easy determination of any marking points, whether it be the case of two pupils or between the two sides of a person's mouth.

Even if a user does not provide the real size information re his/her pupil distance, the method of fitting spectacle frames to a user's face can still be done in a sort of virtual reality setting, in the case where an image of a user wearing spectacle frame is available. The benchmark ruler 32 of the calibration paste-over device 30 can be moved to overlay the user's facial image, so that the two outer edges of a spectacle frame image are aligned to the two outer edges of the spectacle frames as shown in a user image. A second user image, not wearing any spectacle frame, is transmitted to calibration paste-over device 30, so that the second image is aligned to the first image (with spectacle frame), an automated calculated size ratio can be obtained based upon the benchmark ruler 32.

A user's facial image, as acquired via scanner or other means, may include size parameter of the horizontal length and/or vertical height of a spectacle frame worn by the user, in the case where such user is near-sighted or far-sighted, wearing corrective optical spectacles and seeking to have a shelter frame, such as sun glasses. The system is thus able to determine the correct size ratio to fit the shelter frame over the first (optical) spectacle frame and the frontal digital image of a shelter frame to be superimposed over.

In the case where users did not provide any real world size information, the database will build in, as the services are provided to more people, the user statistics for intelligently providing preferred attributes for consumer selection, based upon gender, age, ethnic group, etc. As initial setup, Choice Center 10 of present invention will divide products into senior, adult and children, based upon age. Also, the average pupil distance differs as among Europeans and Asians. Such statistics, as gradually set by users of present invention, will help provide more efficient use of this method of spectacle frame fitting as disclosed herein.

When user information is being entered, the attributes can further include height, weight, shape of face, for advanced indexing. Combining the attributes of gender, age, ethnic group, height, weight, the Choice Center 10 can then present a pre-selected subset of spectacle frames that are suited for the specific user.

After measuring and obtaining the distance between 2 pupil mark points, digital frame images 12 can then be selected to match facial image 21, simulating a consumer's actually test-fitting a spectacle frame in the traditional way. In the case where colors are an important aspect of test-fitting, such as when people are trying on a shelter frame as sunglasses over regular optical lenses, present invention envisions and allows the use of color-filling technique, as in the case of Adobe Photoshop graphics tool, to add to the lenses area, showing the desired color for consumers to see the effect.

When spectacle frame database 11 starts to store the spectacle frame images 12, the frame body, frame legs, nose bridge and pads and lenses (the areas defining lenses) can be separately selected for use as part of digital frame images 12 used for displaying on the computer monitor as users may choose to see only that part over the facial image 21. As such, the CPU 13 is programmed to adjust or crop out some parts of digital frame image, responding to the desired effect stated herein.

This information used and derived herein pursuant to the disclosure of present invention for fitting spectacle frames is also good for the research and development tool for coming up with new eye-wear or spectacle frames, since different parts of a spectacle frames can be selected for combination and showing on a display unit, and for observation and valuation on a simulated person (facial image).

The advantage of present invention is obvious in that consumers and view a huge selection of spectacle frames for the “try on” images of himself/herself, as long as the computer database 11 contain these digital images 12, even in the case where the optical lenses need medical prescription.

The digital frame image 12 in the database 11, as shown in the Choice Center 10 contains spectacle frames having the legs fully opened. A crop-block 323, as used by Calibration paste-over device 30 on image 21 near the, may be in the shape of a rectangle, oval or crescent-shaped. Said crop-block 323, when used on image 21, has a border crop-line that is set to mimic the border facial silhouette line abutting the ear portion, so that a consumer's ear portion on the image 21 can be cut out in the first step of paste-over according to the defined shape of said crop-block 323, and, in the second step, paste-over the digital frame image 12 over the face, including the cropped out ear portion.

In the paste-over process as performed by computer on said crop-block 323 when the side views of facial image and side view of spectacle frame images are being pasted back in the first step stated above, the facial image 21, and the part of the ear of a user and the corresponding ear position, is further divided into three pictures, and secondly leave the ear image of a user facial image on the display device.

As a third step, the spectacle frame side view where part of a leg is covered will be pasted over the position relative to the ear on the facial image of a consumer. As a fourth step, the user image containing no ear or ear shape will be pasted back to the original place. As a fifth step, the spectacle frame side view containing no covered leg will be pasted over the image containing no ear or ear shape on the user's facial image. As a sixth step, the cropped out ear image will be pasted back to its original place.

The crop-away partial image may also include objects that can be seen covering some part of the ears, such as hair, caps or other jewelry items.

In another embodiment of present invention where a user side view image is the only image being worked on for cut-away and paste-over, the simplified steps stated below can be done:

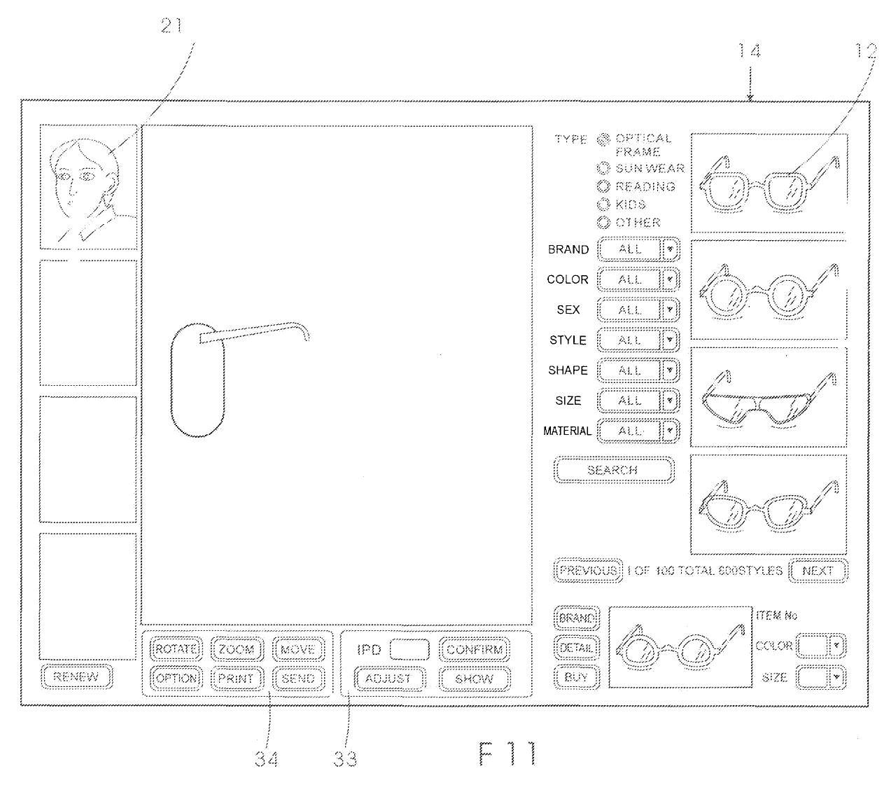

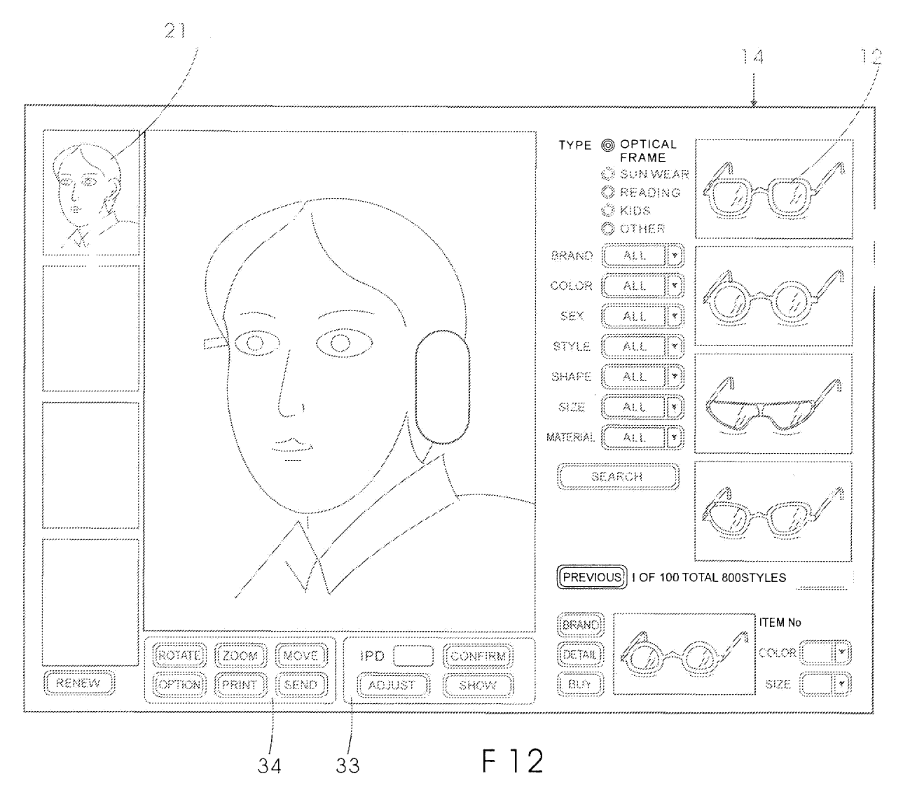

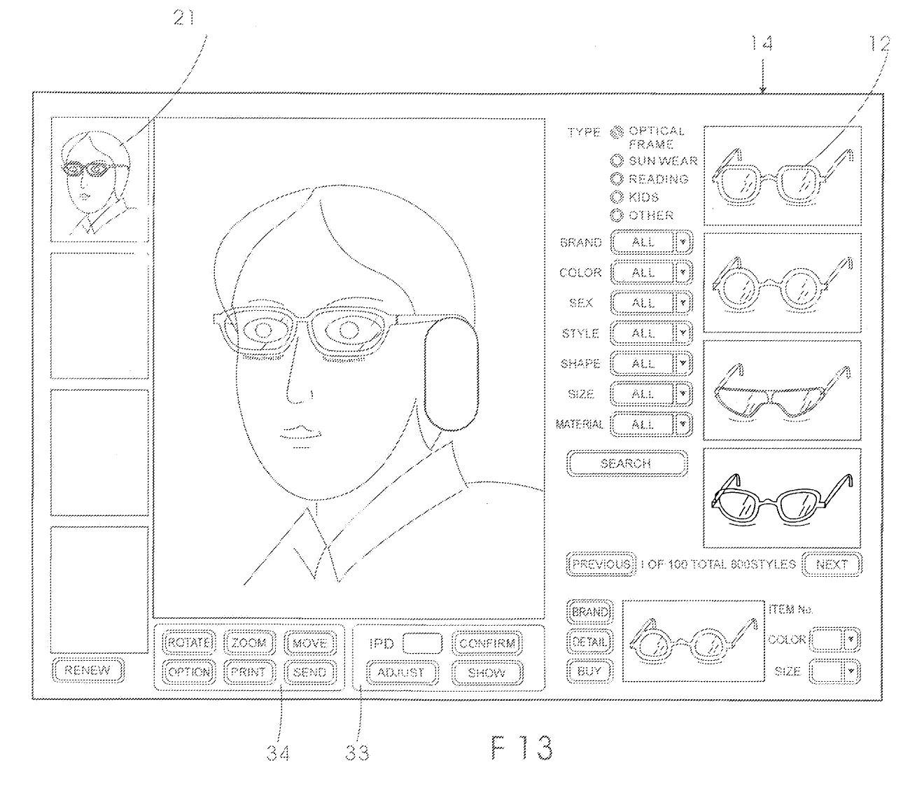

A side view facial image at a pre-selected angle as acquired showing only one ear is presented, as shown in FIG. 9.

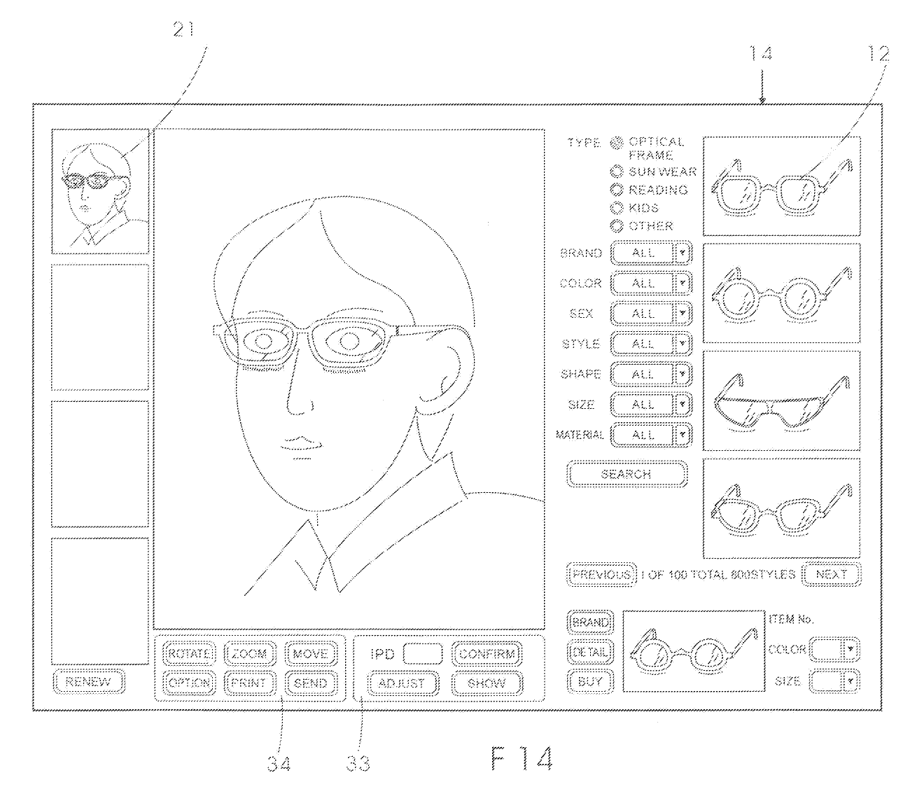

A side view image of a spectacle frame of substantially same pre-selected angel containing only one leg having is superimposed over said side view facial image, as shown in FIG. 13.

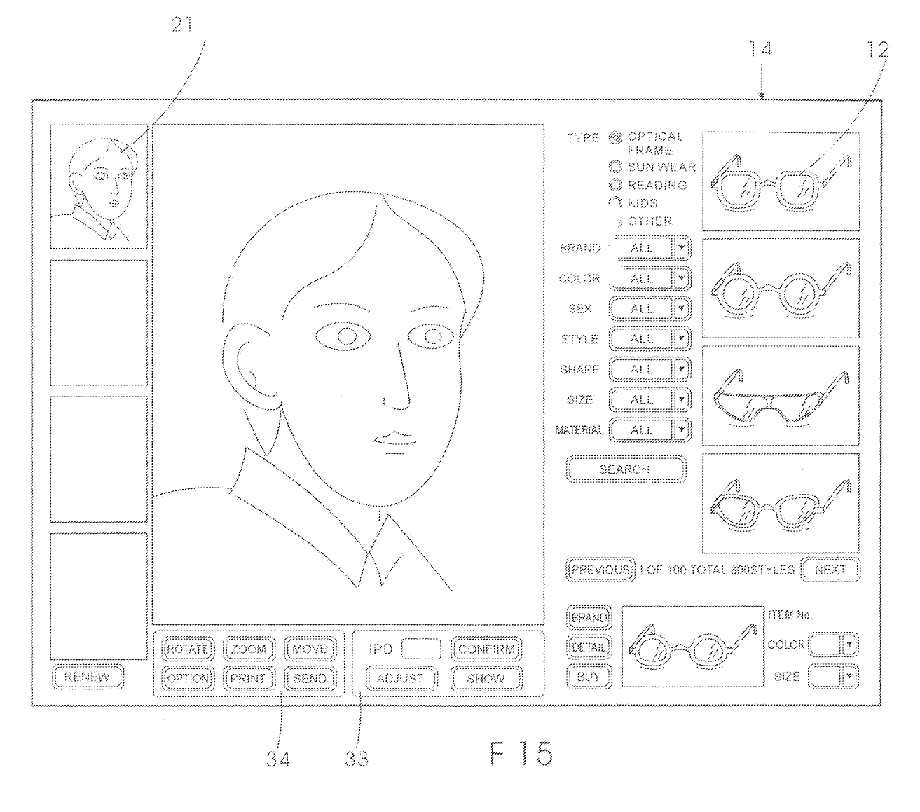

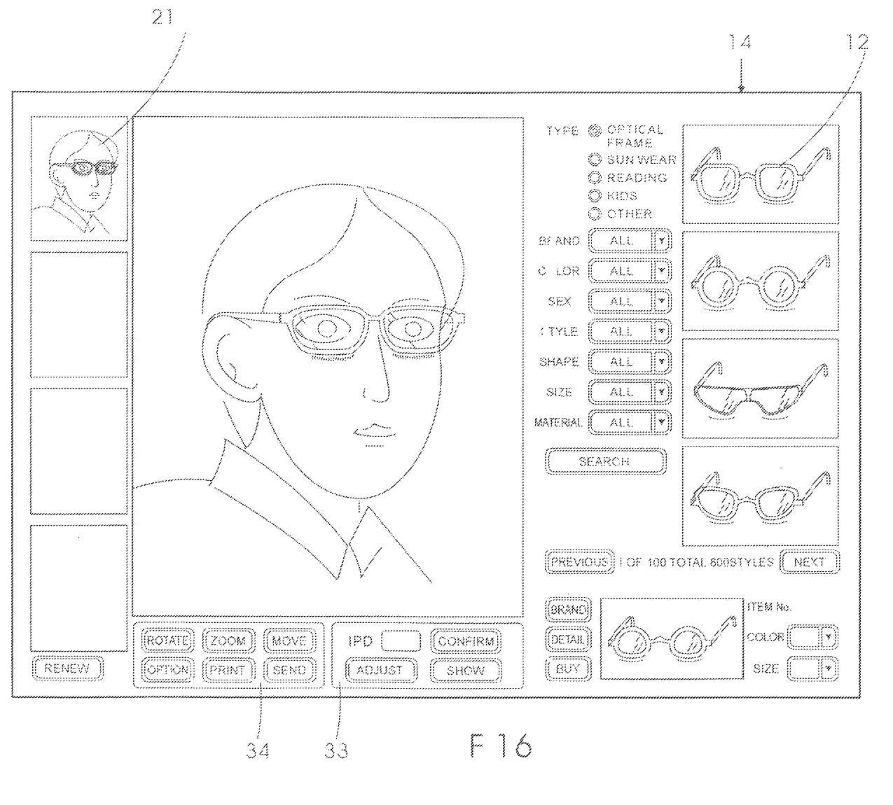

User image having said one-legged spectacle image is further is pasted with ear image, as shown in FIG. 14, resulting in only one side of a user image's 21 face is pasted with the image of a spectacle frame 12.

A common technique, dulling, is used to enhance the “real world” sense of test-fitting when spectacle frame legs are inserted into hair or behind ears, as is available in the popular graphics tool Adobe Photoshop.

Alternatively, the crop-block 323 covering a consumer's ear can be used as the benchmark marking ruler, for use in conjunction with paste-over device 30, allowing user to operate the benchmark ruler 32 in this fashion.

Since computer is taking care of the calculation in the paste-over, the digital frame image 12 can be pasted anywhere on the facial image 21, or along the horizontal measurement line 322, which is normally set to coincide with the line connecting the two pupils of a consumer's face.

The optical products buying process, in accordance with the infra-structure disclosed herein, will greatly promote the customer acceptance to the goods sold this way, since the selling and trying-on process is made more easier. A further use of present method includes making customers into members of a buying club for remotely stored goods and allowing member information database 15 to pre-store customer info, so that the purchasing process is further streamlined, due to the available of spectacle frame information in database 11 and customer database 15 and the ease of advertisement and user feedback.

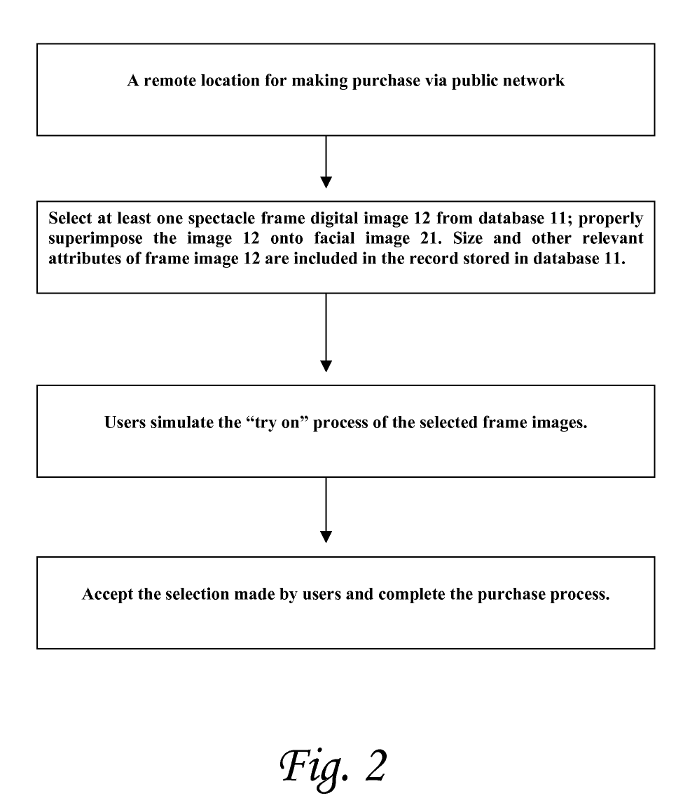

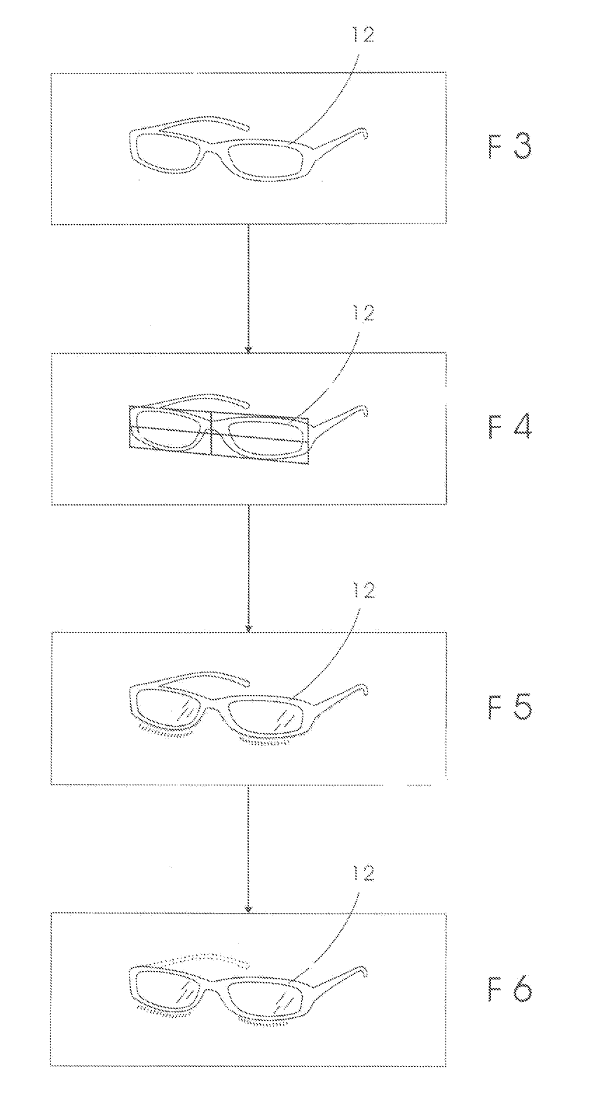

The method of test-fitting spectacle frame over the public network, as shown in the flow chart of FIG. 2, contains the following steps:

(a) provide a Choice center 10 for user's access to public network, wherein said Choice Center 10 contains a database 11 with huge selections of spectacle frame digital images. All frame images come with the associated size information and other relevant attributes.

(b) provide a transmission platform 20 connected to database 11, allowing consumers to transmit their images over public network.

(c) select at least one spectacle frame digital image 12 from database 11.

(d) based upon marking points such as two pupils on facial image 21, calibrate and size the facial image 21 properly to the spectacle frame image 12.

(e) superimpose the spectacle frame image 12 over the facial image 21, resulting in the simulated visual effect of “trying on”.

Additionally, a more streamlined three-step process can also be done to complete the paste-over procedure:

(a) side view facial image at a pre-selected angle as acquired showing only one ear is presented.

(b) a side view image of a spectacle frame of substantially same pre-selected angle containing only one leg is superimposed over said side view facial image.

(c) user image having said one-legged spectacle image is further pasted with ear image.

The spectacle frame fitting and optical products buying process, in accordance the process and mechanism disclosed herein, will greatly promote the customer acceptance to the goods sold this way, since the selling and trying-on process is made easier and more accessible. A further use of present method includes turning customers into members of a buying club for remotely stored goods and allowing member information database 15 to pre-store customer info, so that the purchasing process is further streamlined, due to the available of spectacle frame information in database 11 and customer database 15 and the ease of advertisement and user feedback.

For people reasonably skilled in the art and who follow the teachings and the substance of present invention, it is understood that the embodiments disclosed herein are meant as illustration and not as limitation to the scope. The method as taught herein can be varied, in accordance with the teaching, and still present the claimed advantages and functionality and certainly are part of the scope of present invention.

What is claimed is:

1. A method for 3-D simulation database-driven stored-image spectacle frame fitting services over public network, comprising:
(a) Produce the frontal view and side view of spectacle frames having open frame legs and derive the digital image of said frontal/side views after the background and the optical lens are made transparent and said side view images are based upon pre-set angles of said frame when rotated about the center of said frame, with each of said side view frame images further divided into images showing the legs being obscured as perceived due to the angle of rotation;
(b) Enter the vertical height, horizontal length, central point as marked by vertical and horizontal lines, and other relevant attributes of said digital images when they are stored into computer databases; and,
(c) Set up a calibration paste-over device containing a benchmark ruler having a crop-box on the two horizontal sides of said ruler whereby said benchmark ruler allows two marking points to be selected as reference points relative to facial images when displayed.

2. The method in claim 1, wherein crescent-shaped reflective color effect is added to the opposite and corresponding spots on the two lenses, or the area on the spectacle frame defining two lenses and color-fading effects are applied, resulting the desired effect of light-reflection as can be perceived similar to real life situation.

3. The method in claim 1, wherein the lower portion or the underside of the spectacle frame image is further superimposed with shade-lines and shades of gray, to enhance the effect of shading as can be perceived similar to real life situation.

4. The method in claim 1, wherein the lower portion or the underside of the spectacle frame image is further superimposed with shade-lines and shades of gray, to enhance the effect shading as can be perceived similar to real life situation.

5. The method in claim 1, wherein the relevant attributes of digital images of spectacle frames include pre-selected angles between 0-90 degrees, so that the images having angles between 90-180 degrees are not needed due to image reversion calculation correspond to a mirror line at 90 degree.

6. The method in claim 1, wherein said facial image includes the size parameter chosen as actual distance of two points on a facial image along the length of benchmark ruler, whether the ruler is placed horizontally or vertically.

7. The method in claim 1, wherein said facial image includes the size parameter chosen as pupil distance on a facial image when the benchmark ruler is placed horizontally and through the line connecting the two pupils on the facial image.

8. The method in claim 1, wherein a ruler is attached to a user's face when said facial image is being acquired, showing the actual size of user's face as a result of such use of ruler.

9. The method in claim 1, wherein said facial image includes the size parameter chosen as the horizontal length or vertical height of a spectacle frame as worn by the person whose image is being acquired.

10. The method in claim 1, wherein the benchmark ruler placed on the facial image can be moved, dragged, expanded or contracted when displayed over a facial image on a display device, along with the selected spectacle frames image.

11. The method in claim 1, wherein the paste-over involves the steps of:
a. mimic the border facial silhouette line abutting the ear portion, so that a consumer's ear portion on a facial image will be cut out according to the defined shaped of a crop-block.
b. paste-over the digital frame image over the face, including the cropped out ear portion.
c. the spectacle frame side view image where part of a leg is covered will be pasted over the position relative to the ear on the facial image of a consumer.
d. the user image containing no ear or ear shape will be pasted back to the original place.
e. the spectacle frame side view containing no covered leg will be pasted over the image containing no ear or ear shape on the user's facial image.
f. the cropped out ear image will be pasted back to its original place.

12. The method of claim 1, wherein the paste-over involves the steps of:
a. side view facial image at a pre-selected angle as acquired showing only one ear is presented.
b. A side view image of a spectacle frame of substantially same pre-selected angle containing only one leg is superimposed over said side view facial image.
c. user image having said one-legged spectacle image is further pasted with ear image.

13. A mechanism of 3-D simulate on for choosing and test-fitting spectacle frame over public network, comprising:
a. A choice center containing a computer database of stored front and side images for spectacle frames, the display device is accessible over public network and contains great selections of digital images of spectacle frames;
b. A transmission platform and display device connected to above-said computer database via communication protocol, wherein users may transmit facial images from said platform and have the images shown on the display device; and,
c. A calibration paste-over device that includes a benchmark ruler on the display device and that said benchmark ruler contains two marking points that can be used as the alignment/matching reference points relative to facial images when displayed, and said benchmark ruler can be moved, dragged, expanded and contracted, relative to the facial image, so that the marking points can be set to superimposed upon selected marking points (such as centers of pupils) on facial images, resulting in the correct size calculation of face relative to the stored images of the spectacle frames, based upon the attributes of height and width of spectacle frames that have been pre-loaded into database.

14. The mechanism for 3-D simulation fitting spectacle frame of claim 13, wherein said benchmark ruler may be composed of vertical/horizontal measurement lines moveable alongside a user's face according to marking points selected either vertically or horizontally, aligned to the facial images as acquired by input device.

15. The mechanism for 3-D simulation fitting spectacle frame of claim 13, wherein said marking points of the horizontal benchmark ruler are the pupils and said benchmark ruler can be moved, dragged, expanded and contracted, relative to the facial image, so that the marking points can be set to superimposed upon selected marking points (such as centers of pupils) on facial images.

16. The mechanism for 3-D simulation fitting spectacle frame of claim 13, wherein said marking points of the horizontal benchmark ruler are the outer edges of a spectacle frame already worn by a user and said benchmark ruler can be moved, dragged, expanded and contracted, relative to the facial image, so that the marking points can be set to superimposed upon selected marking points of the outer edge of a spectacle frame image already worn on facial image of a user.

17. The mechanism for 3-D simulation fitting spectacle frame of claim 13, wherein said benchmark ruler in the paste-over device contains an additional vertical/horizontal ruler alongside a user's face to show the actual size of a user's face.
